SEQUENCE LISTING (11 sequences)

SEQ ID NO: 1 - DNA, 17 bp, Artificial Sequence
  /note=“SEQ ID NO 1 synthetic primer”
caacggcggccactatg

SEQ ID NO: 2 - DNA, 15 bp, Artificial Sequence
  /note=“SEQ ID NO 2 synthetic primer”
ccaggcgggctttgc

SEQ ID NO: 3 - DNA, 18 bp, Artificial Sequence
  /note=“SEQ ID NO 3 synthetic probe”
caacgattccgatacaca

SEQ ID NO: 4 - DNA, 20 bp, Artificial Sequence
  /note=“SEQ ID NO 4 synthetic primer”
ttcagcaatggagctttcca

SEQ ID NO: 5 - DNA, 18 bp, Artificial Sequence
  /note=“SEQ ID NO 5 synthetic primer”
caaccccagccgttgaac

SEQ ID NO: 6 - DNA, 19 bp, Artificial Sequence
  /note=“SEQ ID NO 6 synthetic probe”
aagcagcaacaactatata

SEQ ID NO: 7 - DNA, 18 bp, Artificial Sequence
  /note=“SEQ ID NO 7 synthetic primer”
atgatgatatcgccgcgc

SEQ ID NO: 8 - DNA, 16 bp, Artificial Sequence
  /note=“SEQ ID NO 8 synthetic primer”
gccttgcacatgccgg

SEQ ID NO: 9 - DNA, 15 bp, Artificial Sequence
  /note=“SEQ ID NO 9 synthetic probe”
cgtcgtcgacaacgg

SEQ ID NO: 10 - DNA, 11825 bp, Homo sapiens
  gene (1)..(11825): human semaphorin 5A
gctctccgccccgcgctctccgcgcccgctcgccccgcgccgatctacttgccgggaaggcggcgccggtggcggctgctctccctgagcccgctcccgagcgctgctttcccgccgcgggtgggcttcgcagcctcaggccagccgcggcccttggcccgctgcagccccggccctccaccttccccgtgcaggggcggcccggccagtgtcgctcatcccgggacgctcccttctcccacccaggactgccccgcggagctggcttggacacccaactttgccacctcgagggtcgtctctgctgggcgcgaacctgcccacccaccggttggccgcgcgctcggggaccgtgctcgtggcccccaagccggtgcccccattctggaactcagcgagtagggggcggctctggggaagtggcagggggcggctgcagctgctgcctccacttccctagccaggtgctgaagaggatcctcggagccgctctggcccccaggcgctggatgactggcaccagcgctcctcgcacctgtgttggtgtgtgagacttgggctggagtgcccacgtggctgtggagtcagtgtgattcatgattgaggaaacgcgtcctccatcctctctctccttggcactttccacacatgaggagaagaagagcttctgtttagaagacacgtgcccagagtcagaggccccttgcccaccatgaagggaacctgtgttatagcatggctgttctcaagcctggggctgtggagactcgcccacccagaggcccagggtacgactcagtgccagagaaccgagcatccagtcatctcctataaagaaattggcccctggttacgggagttcagagcgaagaatgctgtggatttctcgcagttaacatttgacccaggacagaaagaacttgttgtaggagcaagaaactacctcttcaggttacagcttgaggatctgtctcttatccaggctgtggaatgggagtgtgatgaagctaccaaaaaggcctgttacagcaaaggcaaatcaaaggaggaatgtcagaactacatccgggtgcttctggtgggtggcgaccggttattcacctgtgggaccaatgcattcacgcctgtctgcaccaaccgctcgttgagcaacctgactgagatccatgatcagatcagtggcatggcccgctgtccctacagtccccagcacaattccacagcgctcctcacagctggtggggagctctatgctgctacagccatggattttccaggacgtgatcctgccatttaccgaagcctaggcattttacctcctctccgcacggcgcagtacaactccaaatggctcaatgagccaaactttgtgtcatcttatgacatcggaaattttacctacttctttttccgagaaaatgcagtagagcatgactgtgggaaaacagtgttctccagagctgcccgggtgtgcaagaacgatattggtgggcgcttcctgctggaagacacctggaccacattcatgaaggctcgcctgaactgctcccgtcctggggaagtccccttttactacaacgaattgcagagtactttcttcctgcctgagctggatttgatctatggcatctttaccaccaatgtgaacagcattgcggcctcagctgtgtgcgtcttcaacctgagcgccatcgcgcaggccttctctgggcccttcaagtaccaagaaaactcgcgctcggcctggctaccgtatcccaacccaaacccccacttccagtgtggcaccgtggaccagggcctgtacgtgaacctgaccgagagaaatctgcaggatgctcagaagttcattctgatgcatgaggtggtacagccagtgaccacagtgccctccttcatggaggacaatagccgcttttcccacgtggcagtcgacgtggtgcagggcagagaagcgctcgtccacatcatctatttggccacagattacggaaccattaagaaagtgcgggtacccctgaatcagacctcaagcagctgtttgctggaagagattgagctcttccctgagaggcggagggagcccatcaggagcctgcagatcctgcacagccagagtgtcctgttcgtgggcctgcgggagcacgtggtcaagatccccctgaagaggtgccagttctaccgcacacgcagcacctgcattggggcccaggacccttactgtggctgggatgtggtaatgaagaaatgcacaagcctggaggagagcctgagcatgacgcagtgggaacagagcatctctgcgtgtccgaccaggaatctcaccgtggatgggcactttggtgtgtggtctccgtggacgccttgcacgcacacagatggcagcgccgtgggatcctgcctctgtcgaacccgctcctgcgacagcccggccccgcagtgtggtggctggcagtgcgagggccctggcatggagatcgccaactgttccaggaacggaggctggactccctggacctcgtggtctccctgcagcactacctgtgggatcggcttccaggtgcggcagcgctcctgcagcaaccccactcccaggcacgggggccgggtgtgcgtgggacagaaccgcgaggaaagatactgcaatgaacatttgctatgtcccccacacatgttctggacaggctggggtccttgggaacggtgcacagcccaatgcgggggtggcattcaagctcgccgcaggatctgtgagaatgggcctgactgtgcaggctgcaatgtggagtaccagtcttgcaacaccaacccgtgtcctgagctgaagaagaccacgccctggacaccctggacacctgtcaacatctctgacaacggcggccactatgagcaacgattccgatacacatgcaaagcccgcctggctgatccgaatttgctggaagtgggaagacagagaatcgaaatgcggtactgttctagcgacggcaccagtggctgctccacagatgggctttctggggatttcctgcgtgctgggagatactctgcccacacggtcaacggggcttggtcagcctggacgtcgtggtcacagtgcagccgtgactgcagcaggggcattcggaaccggaagcgtgtttgcaacaaccccgaacccaagtatgggggaatgccttgccttggcccatctctggaataccaggaatgcaacattttgccctgcccagtggatggcgtgtggtcttgctggtccccctggacaaaatgttcagcaacatgcggcggtggacactatatgaggacccgctcttgctccaatccagccccggcctatggaggggacatctgcctggggctgcacacagaagaggcactctgcaacacgcagccctgcccagagagctggtcggagtggtcggactggtctgagtgtgaagcctctggcgtccaagtccgcgcccgccagtgcatcctcctgttccccatgggcagccagtgctccgggaacaccacggagagccggccgtgtgtgtttgactctaatttcatcccagaagtatctgtggcaagatccagtagcgtagaagagaaaaggtgtggagagttcaacatgttccacatgatcgccgtggggctgagcagctccatcctcggctgcctcctcaccctgctcgtctatacttactgccagcggtaccagcagcaatcccacgatgcgactgtcatccaccccgtctcacctgccccccttaataccagcataaccaaccacatcaacaaactggacaagtacgactcggtggaggccatcaaggcatttaacaaaaacaacttgatcctagaggaaagaaacaaatacttcaacccacatctcactgggaagacctattctaatgcctactttacagatctcaataattatgatgagtactaacagctttcatgtttttggcttcttgtaaatccccagttcctcaaggcctgtgccccatgactgcccatgtttctgaggcttcagagtcgaagtttggatacatttcaagtgcatttcaagccaccagagtgtcccattgctgccaaaaatacacgtctttaaaagcaacaaaaattgaaataagacatcgtgaaaatcttgaccattgttgaatgagccagggtgtgaagtttttaattgtgttcatcctatttttctgacaagtccattggtttgttttttgagcattattttataaatgtgccacccacattggaaggagtctttctttagaactttggagtgtaaatcttcatgatgttgtaattcaagaaaataggcactttctctgaaagacctgctccttccacaagaagtgcataggtccataatatttcataaaatgaagaaaaagaatgtggccaaacaattattcaccatggattgcccaactttccaaatctggataaagctgtgggattcttggaagcagcttgagtgttttcatcttgcctgggaagcccaggaattccacctggtccacaccggcagaagttacagtagactgtgaggcaccccaccttgctcctgatgcagtttctgtgccattgctggtgctggtggaggcagcggagcagaggctcaggcacaatgaagcgtggatgtgttctgcaggttgctgcaaaactcaccttattctgacttttggatttcatggcattccaggaagctccttgccatgctgttggcctggaagtccacctgtctggtccatagtgacgtcctgaagagccagtctgtaaaataaccaaccacttacttagcgtttggatagactccatgccttctctctccctgcaaagaaaaatcttgaacatttatgatgtcaattagtgaaagtattgaaaatactaaattataactaaaagcaactttttatgttattgaaaatattgaaagaactgatattaatgataatcattttatttttcatctctctgatatacccaatgtgtggcaatcagcccctaccacgagcattaatgccatgtaaagctggctttctggagtctgccaggtccacacgaagtgctacccccagtttctgctgttacttgctgcctccaggccaggggagcaggagatgctcagctctggtgccttttcttttatttcagtgctgccttcccacccacccagtccattcaccctcacctctccctgcatggaggcaacagcatttcaagatgtaccttgggaagtcaggatagctggaagtaagggtttttgggatccctgtggtctcttcactgatcatcgataagcatttaagagtgtgctaacaccattcacagaggtccctggaaaaaaatatataattttctcacaaacaaccaatacataacctatggcagttggttgaataattattgaaaaaaagaacacgactgaaaaagttcatctgatgcctctgagcatgtgataaagtcctgggtagacacggaaacgtgttcttacaaatgaaccttcggatctatgaaagaaaatatagtagggggactaaggaacatagaattttattagcatatgtgattttacccttatctttgtttctaatttaaagaaacaatttcagaaagtgttagaagaattctatgtttaataatgaatattgttgagttcaaaatattctcatatacccagatttacagagatgacacagtattgaaatggcaggtgggctctgtaagttattttggattaatgacattcagggtctttgcagtggggacttcatgttgcctctcactcattcctcatatgtcagagtcttctgtctatatgcacatgctcaaagtccatactcagtaggggaaatctagccggatggttctccaattgttcctcggctggattttcatcatcagatatttcaactcatctcatcagtttcttgtaagaaaaaaacattttcactttgtagcacattttagttatttttgagtcttcttccctaatagtttgtaagcttaaagggacattttattttccctggtaaagaggattctaatatttcaagaatgtttcactggaattgatgggaaatggtttctaaatggcatcgaagctggtctttaatgagtcttttgcaatgacttgggaaaaacaccctccctcactacaggatcttcatgttgttaataatataaacaaacctttgaaattagcattgcaaaagaatcccatttttgtttcgtaccacactgttcaaaggaaaattgtttatctctcttcctttctctctctctcttatttgacagaatagcaagtgtttagataatttcagatatattctaagtattttaccagctgtggaataagttgtgtttgtccatcactgtgtagctaccgttcacatgttctggctctgtactccacgcttatcgcgtgaaccctcacatgttggactttcacagtgtgatctctcacctgtcttggaaactccttcataaagccgctcttccttaggcctgggctgtttggaagtcctggtgaaactttgcggttcacattttaaattcctgaatgaccttttcattctctctttctttatctttgtttttgtccttcattccctccttttgttcttccttcctgccttcttttcttctttcctttcttctttctttaattcttcctttcttcctattttcttggtacaagtggaacaaaaaccaacacatagttttaagctacacctttctgcacctgatgtaaaatgacattaatcactatttaacttctaaaattatttctgaatacagttggagataggctggttttcatgaggaagctggcttggctttagtcttacatttaaattctttgaaagtggctcccagtgctattcagggtcctttcttgaggccagactcatcaccatctactattgattttacagtgcactgacagtttacaggaaggagaagaacagaatttctgcacactacacaacatgtgggcttcctgtagctccagggacatgtagctttggtgaaactggggttttgtaacctctgaatgatatggacttagtgaattctaaggaactcagggggcacgtggtcaggctccaccgcacagaagagccacagtctccagactcatggcgttccctccagaactccccattcccctctgagcatatttctatgctgctgcttcttgtgacttacatgaggcatctcagcttcttcatgtttgtagggatggttccctaagcctgttcaagtaggctctcactagagttaccattcatatttgagaagaaaagaacatttaataaatgtatgtgtggggctgaggttctaaaggaattgaaaagagacgataaacattgaatgagggtgagggcatccctgctggggagaaacctctgtccctaggaagagtccgttcatgtcatgtggtttgggatgaaccaggggtctggccccatcgggtcacaggtgatggcaaatagaaaagaagcaaatggaggaaatagtcggataagtaggtgacgtgaaaggcaggacctggtcaccccagcaagtgctatggacagttcccggaaacggttgcccacttcacaggtccatgggtctgacccttggactctgccaggatcaactgcccagagtgccagagttttagccaaaggtgtacttacttccttatttatctgcaaaaggatggaaactgtgggagtcaaagcctattttgctgagtgttcccactggagtctctggtagaattagcaggtcatgctgtcaaaatcatggacaaaggctgggtgcagtggctcatgcctataatcccagcactttgggaggccaaggtgggcggatcacctgagctcaggagtttgagaccagcctgggcaacatggggaaactccatctctacaaaatatacaaaatattagccagccatcgtggtgcgtgcctgtggtcccagtttcttgggaggctgaggcgggagtatcatttgagccaggaggttgaggttgcagtgagctgagatcacatcactgcactccatcctgggtgacagagagagaccctgtctcaaaaaaaaaaaaaaagaacaaagtcttagcggggggtctctatgtccccaagtggctctctaaggggatccattagcgtataattgaatatcaaagggaattttaccttaaacatatatcaagtctatactcagctaatcgttttgtctaaacacagccttgttagcatttgaatttacaaatattcatttgcttatttgcagtgttccttttgcttttagaataaatcataaatatttccccaatactgtcaagatacttcttcaactttgtccctgaaccatcagctttctttagggtccgggtatcctttacacttcaatggggttttcttgtgtgtgtcattgggggtaactccattgacctcagcatttctatctcaagagatggctctggaacacagaaccagaggcaacaggtactaatggttgataaaattaacagcgactgcataaccattttccacatggcagggctgtgcttaagccccacacctccaataatcaatttctatgacggtactaataaccttaactatatgcaaattcttcaccacttatgatatggtacaaactggagtttacagatggccttggccttgcctagcccatttgttcatcctaacttcttcatcacttcctgatgcacttggggaagcatgttgacacaacgttaccaatatttgattacctatggttatctgcaattacctacgcttatcttatcagaccctagatgccatttgagccccttacttctttgggcagtgggtgaaatgggagaacatagagaaatgttcactttgacctaagaaacgcataaaagggaatgccatgcacaatgtaagataaaatgtttggggacaggggatcatcgtgaaatttatttctgctattctgccctattgaggtcattaattataatggatttaaagatttttttttaaactctacaaacactaagccaaggaattcaggttatctgctatgttaacctaataaagcaaaggaaaataagttgctttaatccttgcttgttaacctaataaagcaaaggaaaatatgttgctttaatccttgcttcttttctcttttggtaatagaaaaggtaaataagatgtaatgttgaactgctggtgatgatttctaaaacgactctttaaatactcctttcctatggacttatattaaagatgcattgtacacattgttttagcagatagatcaaagatatactcagtggtcatctgttgcattatcagaaatttgatacatgttacagaagcatgaggaagggaatcctatccacttttgaaaatagagttcacattgcaagcgacatagggcattatcaaaaccaattcagagcctggcacaaaatgtatacttcctagtgaaagctgtctcaaaacacagcctttttgatggatcatattagctcatatctgccatagaaaggcaatcaagcttactccctgcaatcggagtcaatatcaattcttgctcaagcaaacatgttttacagtgttggcgcaatggcaggaactagtcaagggcactttcagatctggaaagaggaaagaggaggtagaaaatcactcttttgtgtttcttaaactgtcaatcaagcaagaaaatttattccaccccttcatatttgctctaaaatctggggccagtagttgaaatgtgatcttgttctttctcagccttttggcaatttaatgccaaatattccaaatgttagatgaatggcagtaataaaatgattcaaatgtttgataaatacaagctgagagcaaaatctggactctgaaaaatcggaactattttatcatgttgctaaaatgagagcatcattttcttccctctctgtaagtgcggtagtttaatttcctagaaaaagttgctagtgccttgtttaagatgatcatttatcatttcatgtggatattattggtctaattagaaggagaagattgattggatttactttaaagaaaattattctcctatgtctctttgaactcaggaatagaaaatgaccaagaagcacatcatcttcagatgtgatttttgccctcactggggatgtagaaccagaataggtacaacatatgctcagaagagaggtgacttcagtattagccttctagaaactaacatcactttaatttttacatgttccaaaaaaaatcatagtgattgttctgtaataagaatgttaacgtgatgcctatagaagttagaggtttttgtttctgtttctcttcaggcaagtccatagatagaggaaaatcagacacaggctggtgacaactggtatagatacagtgagcatggaatgagaaccacctacaacattgcctgattatttttaagtttatatcaaaactgcttcacaagttcacaaaagttgtcactcttaatttgggttaagaaaagtaatagtattatctctgctgaagttgattgtttttctcttttaccatgactgttggccaaaagcaagctgaacctctgcactttgaaacaaagcacagtatgaatctttaacccaagcacttagtttcaccactgagattctacatcatctgcaagaaaatgcacaaacttttggtccaatgttggattcttcaaaagtgcatagcaaaacttttgtgctatgccgcttgatgtaacttttaaagatgttatcaaatgttaaatgcctcattctgcgttaatttgctgttggctgttttgtgaacacaaaataaacttgagatttttttttttttttttttttgtgaaactcctccaaagccaatttcagcccttggtaagcttgcagacagttactgatcttaaatttaattttaaaagacaatgtatcttaattataatttagctttttaaaacaatgagatagctttacatttcccctttgtttgaatgagaaaatggatcttgggttgctatgctagaacacttgtagattgctgggtcctttgtaagggggccatggacacaccacactttctttcaatccttacatttgaagcattgatattcttcaaaaccttcttgttacatgtgcgcaatagaaatttctaatgttcatgacttttatctttcctgtccatcaattcactggttgtaaatgcttcctgagagctgtctaggtctgtatcccagattgttgcttaatgacatctgacagatgcattgttttctgaaatcagcttaagacaccaattgtggcaactggaaactcattacctgctgcattggatcaactatggaagttggagcaggggtgggcggaggtcacctaaccaatcaatggaaggcaactcacacctgctccaagcctcagctttgagaaacaaacacgtttataagaaaaaatatatagctattattacagaagtgaatatgttgtgctctcttactgctcttggtgcattgacagtttctgtatctcaaccctattcatctttatgaaaaagcattctgaagatctatcctcagcactgctgagtgtgcagtcacactttcctaccaacccccttcttaccatctctagctgccatttgtggggggaacaaaacaagggaatcctgattgtgtacagtataaattgtattatattttttgtacttatgttttatgtaaatagtttgtctcattcaattgtatgtgagcattgaaataaatcctaccatttaggggacaatttaaaaaaaaaaaaaaaaaa

SEQ ID NO: 11 - protein, 1074 aa, Homo sapiens
  PEPTIDE (1)..(1074): human semaphorin 5A
MKGTCVIAWLFSSLGLWRLAHPEAQGTTQCQRTEHPVISYKEIGPWLREFRAKNAVDFSQLTFDPGQKELVVGARNYLFRLQLEDLSLIQAVEWECDEATKKACYSKGKSKEECQNYIRVLLVGGDRLFTCGTNAFTPVCTNRSLSNLTEIHDQISGMARCPYSPQHNSTALLTAGGELYAATAMDFPGRDPAIYRSLGILPPLRTAQYNSKWLNEPNFVSSYDIGNFTYFFFRENAVEHDCGKTVFSRAARVCKNDIGGRFLLEDTWTTFMKARLNCSRPGEVPFYYNELQSTFFLPELDLIYGIFTTNVNSIAASAVCVFNLSAIAQAFSGPFKYQENSRSAWLPYPNPNPHFQCGTVDQGLYVNLTERNLQDAQKFILMHEVVQPVTTVPSFMEDNSRFSHVAVDVVQGREALVHIIYLATDYGTIKKVRVPLNQTSSSCLLEEIELFPERRREPIRSLQILHSQSVLFVGLREHVVKIPLKRCQFYRTRSTCIGAQDPYCGWDVVMKKCTSLEESLSMTQWEQSISACPTRNLTVDGHFGVWSPWTPCTHTDGSAVGSCLCRTRSCDSPAPQCGGWQCEGPGMEIANCSRNGGWTPWTSWSPCSTTCGIGFQVRQRSCSNPTPRHGGRVCVGQNREERYCNEHLLCPPHMFWTGWGPWERCTAQCGGGIQARRRICENGPDCAGCNVEYQSCNTNPCPELKKTTPWTPWTPVNISDNGGHYEQRFRYTCKARLADPNLLEVGRQRIEMRYCSSDGTSGCSTDGLSGDFLRAGRYSAHTVNGAWSAWTSWSQCSRDCSRGIRNRKRVCNNPEPKYGGMPCLGPSLEYQECNILPCPVDGVWSCWSPWTKCSATCGGGHYMRTRSCSNPAPAYGGDICLGLHTEEALCNTQPCPESWSEWSDWSECEASGVQVRARQCILLFPMGSQCSGNTTESRPCVFDSNFIPEVSVARSSSVEEKRCGEFNMFHMIAVGLSSSILGCLLTLLVYTYCQRYQQQSHDATVIHPVSPAPLNTSITNHINKLDKYDSVEAIKAFNKNNLILEERNKYFNPHLTGKTYSNAYFTDLNNYDEY

(19) United States
(12) Patent Application Publication
(10) Pub. No.: US 2011/0293560 A1
(43) Pub. Date: Dec. 1, 2011

(54) METHOD FOR DETERMINING IMMUNE SYSTEM ACTIVITY AND MEDICAMENT FOR INFLUENCING SAME

(75) Inventors: Rainer Blasczyk, Hannover (DE); Christine Gras, Hannover (DE); Constanca Ferreira de Figueiredo, Wedemah (DE)

(73) Assignee: MEDIZINISCHE HOCHSCHULE HANNOVER, Hannover (DE)

(21) Appl. No.: 13/099,185

(22) Filed: May 2, 2011

(30) Foreign Application Priority Data
Apr. 30, 2010 (EP) 10161687.8

(51) Int. Cl. A61K 38/20; A61P 35/00; G01N 33/566; A61K 39/00; C12Q 1/68; G01N 33/53

(52) U.S. Cl. 424/85.2; 435/6.12; 436/501; 435/7.92; 424/185.1

(57) ABSTRACT

A method for the determination of the state of the immune system, e.g. the state of activity and activation, respectively, of the immune system, using the determination of presence of a blood or peripheral mononuclear cell or plasma marker. The marker can be determined directly, e.g. by determination of its titre using a specific antibody against the marker, or indirectly, e.g. by the determination of the mRNA encoding the marker. Further, the invention relates to the use of the protein corresponding to the marker analysed in the analytical method, as a medicament, especially for influencing, e.g. for activating the immune system, more specifically for activating the cytotoxicity of NK cells as well as for activating the cytotoxicity and the proliferation of T-cells.

### PRIORITY CLAIM AND REFERENCE TO RELATED APPLICATION

This application claims priority under 35 U.S.C. §119 from EP 10161687.8, which was filed on Apr. 30, 2010.

### BACKGROUND

Generally, for diagnosis of the GvH disease, which is indicated e.g. by skin rashes, an organ biopsy is generally used.

Paczesny et al. (Science Translational Medicine 2, 13ra2 (2010)) described the analysis of Elafin as a specific biomarker in plasma for the graft versus host disease.

US 2005/042636 A1 describes that abnormally proliferating cells, i.e. tumour cells, overexpress the Sema5 gene product and accordingly SEMA5 antigens can be used to generate an immune response against tumour cells, e.g. SEMA5 specific cytolytic responses and protective antibodies against SEMA5 as well as Sema5 antisense nucleic acids to inhibit expression of Sema5. For SEMA5A, cell membrane localization was verified.

WO2005/104785 A2 describes a medicament for the treatment of liver cancer containing as the active agent an antibody directed against a membrane-associated peptide, e.g. prostaglandin receptor membrane component 1, a membrane-bound transporter (SLC2A2), or SEMA5A, as these peptides have been found to be up-regulated in tumour tissue compared to healthy tissue.

The state of art uses SEMA5A as a tumour marker or as an anticancer target, respectively, but not for determining the activity of the immune system.

Generally, it is known that tumours necessarily evade being recognized by the immune system, i.e. tumours are not recognized as such by the immune system, and therefore tumours naturally do not raise an immune response.

### SUMMARY OF THE INVENTION

The invention provides a method for analysis of the state of activation of the immune system of a patient comprising determination of the concentration of Semaphorin 5A in a sample of the patient. Preferably, the concentration of Semaphorin 5A is determined by detecting the mRNA encoding Semaphorin 5A, or by detecting the reaction of an antibody that is specific for Semaphorin 5A protein. A Kit of components for use in a method of the invention includes a pair of oligonucleotides specifically hybridising to a nucleotide sequence containing at least a fraction of the nucleotide sequence encoding Semaphorin 5A. A medicament of the invention for activating the immune system in a patient is Semaphorin 5A protein in a pharmaceutically acceptable formulation. A process for the production of a pharmaceutical composition which is for use in the activation of the immune system of a patient, excluding cancer patients, of the invention is by providing Semaphorin 5A protein in a pharmaceutically acceptable formulation.

### BRIEF DESCRIPTION OF THE DRAWINGS

The invention is described in greater detail below with reference to the following figures:

FIG. 1 shows mRNA levels of Semaphorin 5A in PBMCs in allogeneic hematopoietic stem cell transplant recipients,

FIG. 2 shows mRNA levels of Semaphorin 3A in hematopoietic stem cell therapy patients,

FIG. 3 shows a Western blot for Semaphorin 5A protein in patient sera,

FIG. 4 shows concentrations of Semaphorin 5A protein in patients prior to and after hematopoietic stem cell transplantation,

FIG. 5 shows concentrations of mRNA of Semaphorin 5A, normalized to β-actin mRNA levels, in healthy humans and in patients after hematopoietic stem cell transplantation,

FIG. 6 shows concentrations of Semaphorin 5A protein in healthy humans and in patients after kidney transplantation,

FIG. 7 shows concentrations of Semaphorin 5A protein in patients diagnosed with rheumatoid arthritis and in healthy humans,

FIG. 8 shows concentrations of Semaphorin 5A mRNA in patients diagnosed with rheumatoid arthritis and in healthy humans,

FIG. 9 shows the recombinant Semaphorin 5A detected by Western Blot after purification by affinity chromatography,

FIG. 10 shows the upregulation of the activation marker NKp44 NK cells upon stimulation with Semaphorin 5A,

FIG. 11 shows the upregulation of activation marker CD25 on NK cells upon stimulation with Semaphorin 5A,

FIG. 12 shows the expression levels of NKG2A and NKG2D receptors on NK cells upon stimulation with Semaphorin 5A,

FIG. 13 shows the upregulation of Granzyme B mRNA in NK cells induced by Semaphorin 5A,

FIG. 14 shows the effect of Semaphorin 5A in increasing the cytotoxic potential of NK cells against target cells,

FIG. 15 shows the effect of Semaphorin 5A on the proliferation of CD8⁺ T-cells, and

FIG. 16 shows the upregulation of Granzyme B in CD8⁺ T-cells by Semaphorin 5A.

In the figures, p<0.05 is indicated by *, and p<0.01 is indicated by **, which is both considered as statistically significant in the Mann-Withney U-test applied in the statistical analysis. In the figures, the same abbreviations denote the same samples or experiments, respectively.

### DETAILED DESCRIPTION OF THE PREFERRED EMBODIMENTS

The invention provides an alternative diagnostic test method that can reliably determine the state of the immune system, e.g. a state of the immune response indicative of the GvH disease, transplant rejection, including organ rejection, and indicative for the HvG reaction (HvGD), or indicative of an autoimmune disease. The invention also provides a compound for use in the activation of the immune system.

The present invention relates to a diagnostic method for the determination of the state of the immune system, e.g. the state of activity and activation, respectively, of the immune system, especially of Natural Killer (NK)-cells and of T-cells, using the determination of presence of a blood: peripheral mononuclear cell or plasma marker. The marker can be detected directly, e.g. by determination of its titre using a specific antibody against the marker, or indirectly, e.g. by the determination of the mRNA encoding the marker.

Further, the invention relates to the use of the protein corresponding to the marker analysed in the analytical method, as a medicament, especially for influencing, e.g. for activating the immune system, more specifically for activating the cytotoxicity of NK cells as well as for activating the cytotoxicity and the proliferation of T-cells.

The diagnostic method is especially suitable for use in the determination of the status of the immune system in samples obtained from patients having received a transplant, which especially is an allogeneic transplant of stem cells, e.g. of bone marrow stem cells in order to determine the state of the graft-versus-host (GvH) disease. Further, the diagnostic method is suitable for determination of the status of the immune system in a sample obtained from a patient having an autoimmune disease, or for monitoring the state of activity of the immune system in a sample obtained from a patient who is receiving medication influencing the activity of the immune system.

In a second aspect, the invention especially relates to the activation of the immune system by making use of the characteristics of the protein to activate the immune response, e.g. in an immuno-compromised patient. Therein, the invention relates to the compound for use as a medicament or for use in the manufacture of a medicament, especially for the treatment of a low activity of the immune system.

An analytical or diagnostic method is provided, and an analytical or diagnostic test kit suitable for performing a method of the invention, which analyses the concentration of Semaphorin 5A, either of Semaphorin 5A (SEMA5A) protein or peptide, e.g. by detecting the reaction of an antibody having specificity for SEMA5A in a human patient sample, and/or of Semaphorin 5A-specific mRNA. In this first embodiment of the invention, there is provided a method for determining the state of activity of the immune system by determining the concentration of Semaphorin 5A, either by determining the concentration of Semaphorin 5A protein, and/or by the determining the concentration of Semaphorin 5A-specific mRNA, wherein the concentrations of Semaphorin 5A protein or mRNA preferably are determined in relation to the concentration of Semaphorin 5A protein or mRNA determined in healthy humans, and/or in relation to the concentration of a housekeeping gene product, i.e. protein, or mRNA thereof. Preferably, the concentration of Semaphorin 5A in the patient sample, preferably also in a sample of a healthy human, is determined in relation to a constitutively expressed component of the sample, e.g. the gene product of a house-keeping gene for normalization, and/or the concentration of Semaphorin 5A in the patient sample is determined in relation to the concentration of Semaphorin 5A that is determined in at least one sample of at least one healthy human. More preferably, the method further comprises the step of correlating the concentration of Semaphorin 5A determined in the patient sample to the concentration of Semaphorin 5A determined in the sample of at least one healthy human, and most preferably the method comprises the step of assigning an elevated concentration of concentration of Semaphorin 5A in the patient sample over the concentration of Semaphorin 5A in the sample of the at least one healthy human to an activated state of the immune system of the patient.

In a second embodiment, the present invention provides for Semaphorin 5A for use in the production of a pharmaceutical composition, and Semaphorin 5A for use as a pharmaceutical active agent of a medicament in the treatment of a lack of immune activity, i.e. for increasing the activity of the immune system, especially for increasing the proliferative and cytotoxic activity of NK cells, and for increasing the proliferation and the cytotoxic activity of T-cells, especially CD8⁺-T-cells. In greater detail, Semaphorin 5A is provided for use as a medicament, especially as an agent for stimulating and/or activating the immune system, which activation of the immune system preferably comprises or consists of activating NK cells, increasing the cytotoxicity of NK cells, and increasing the proliferation of T-cells, especially CD8⁺-T-cells. Preferably, Semaphorin 5A is for use as a medicament in combination with IL-2 and/or IL-15. Accordingly, this embodiment also relates to a medicament comprising Semaphorin 5A which is adapted or prepared for the medical treatment, especially for the activation of the immune system.

The patients from which the samples originate that are tested for the titre of SEMA5A, and the patients receiving SEMA5A as a medicament preferably are transplant patients and patients having been diagnosed or being suspected of having an autoimmune disease, and preferably excluding tumour patients. Preferred transplant patients are recipients of one of the group comprising or consisting of allogeneic hematopoietic stem cells, solid organs or tissue, e.g. kidney, liver, heart, lung, skin. Preferred autoimmune diseases are comprised in the group containing or consisting of lupus erythematodes and rheumatoid arthritis.

The embodiments of the invention are based on the observation that the titre of Semaphorin 5A and of Semaphorin 5A-mRNA is increased in patients being suspected of GvH disease or having clinically manifest GvH disease by a factor of about 20 in comparison to healthy patients. Further, it has been observed that the immune system can be activated by administration of Semaphorin 5A protein, as e.g. exemplified by the in vitro activation of the cytotoxic activity and increased cytotoxic activity of NK cells by Semaphorin 5A, and as exemplified by the proliferation of CD8⁺ T-cells upon addition of the recombinantly produced Semaphorin 5A protein to the cultivation medium. The activation of NK cells by presence of SEMA5A could also be shown by induction of cytokine secretion upon stimulation of NK cells with Sema5A in comparison to the cytokine secretion profile of non-stimulation NK cells, e.g. induction of increased secretion of IL-1β, IL-6, and IL-8. Furthermore, a significantly increase in the secretion of IFN-γ, IL-1β; IL-6, IL-8, TNF-α, GM-CSF was measured when NK cells were incubated in the presence of SEMA5A in combination with IL-2 and IL-15 in comparison to the cytokine levels detected in the presence of this cytokine combination alone.

It was found that in patients having received a transplant, e.g. an allogeneic kidney or allogeneic hematopoietic stem cells suffering from acute transplant rejection or GvHD, respectively, and in patients having an autoimmune disease, e.g. having been diagnosed with rheumatoid arthritis, an increased concentration of Semaphorin 5A, determined both as protein and mRNA, correlates with the disease. In patients who suffered both from chronic GvHD and from cancer, e.g. thyroid or lung cancer, and who underwent anti-cancer therapy the correlation of the increase in the concentration of SEMA5A to the GvHD or autoimmune disease was found to a smaller extent. Accordingly, the analytical method of the invention preferably indicates the presence of an acute GvHD or HvGD, and an acute phase of an autoimmune disease.

Semaphorin 5A as the analyte of the method of analysis is not necessarily bound to cells, but can also be determined in cell-free samples, e.g. in blood serum. The sample used in the analytical method can therefore be selected from blood, including whole blood, blood cells, e.g. PBMC, and preferably the sample is serum, i.e. the cell-free component of blood.

For identifying a marker that is suitable for identifying a status of activity of the immune system indicating GvH disease, approximately 60 patients having received hematopoietic stem cell therapy (HSCT) using allogeneic stem cells were monitored for the GvH disease (GvHD), i.e. the activation of the immune cells was monitored. In accordance with standard practice, this was done by a biopsy, because no validated diagnostic blood test is available that can determine the graft versus host disease, i.e. an activation of the immune system in response to antigen.

When searching for a biomarker that is indicative of the activation of the immune system or occurring in the GvH disease, it has been found that the level of Semaphorin 5A protein in peripheral blood is a reliable marker for the presence of an increased activity of the immune system, i.e. for the immune response of the graft versus host disease in the transplant patients.

It has been found that levels of Semaphorin 5A, e.g. determined as Semaphorin 5A mRNA, showing an increase by a factor of at least 2, preferably by a factor of at least 4 to 5, preferably by a factor of at least 10, more preferably by a factor of about 15, and most preferably by a factor of about 20 or above in relation to an average concentration of Semaphorin 5A indicate an increased activity of the immune system, which is e.g. indicative of GvH disease in hematopoietic stem cell transplant recipients, or of an autoimmune disease. Accordingly, the diagnostic method preferably includes the step of normalizing the signal specifically detected for Semaphorin 5A to a constitutively expressed gene product, and determining the relation of the normalized signal for Semaphorin 5A to the signal for Semaphorin 5A which has been determined and normalized in a sample obtained from a healthy person. The resulting relation of the signal for Semaphorin 5A of the patient to the signal obtained in a healthy person can be edited, e.g. for subsequent medical evaluation.

Generally, Semaphorin 5A, or the mRNA encoding Semaphorin 5A, can be analysed by the method in a sample obtained from a patient, which sample is preferably a cell-containing sample, e.g. blood, and specifically peripheral blood mononuclear cells (PBMC).

Preferably, the concentrations of Semaphorin 5A are standardized by relating them to the signal determined for a constitutive sample component. For embodiments of the method for analysis determining the concentration of Semaphorin 5A by RT-PCR, i.e. by reverse transcription of RNA isolated from the sample to cDNA, followed by specific amplification of a fragment of the semaphorin 5A coding sequence by PCR, the O-actin encoding sequence can be the constitutive sample component, in relation to which the signal specific for Semaphorin 5A is normalized.

As an alternative to determining the mRNA by reverse transcription of RNA to produce cDNA, followed by PCR on the basis of the cDNA using primers specific for the semaphorin 5A coding sequence, a specific antibody reaction against Semaphorin 5A can be used. In this embodiment, cell-free serum can be used for the analysis of Semaphorin 5A, especially for the determination of Semaphorin 5A protein, e.g. using an antibody having specificity to Semaphorin 5A protein in an ELISA. Polyclonal, preferably monoclonal antibody that is specifically directed against Semaphorin 5A can be produced by standard methods, e.g. by immunizing an experimental animal with Semaphorin 5A and collecting serum for polyclonal antibody, or using spleen cells from the immunized animal for generation of hybridoma and selecting a clone to produce a monoclonal antibody.

When analysing the activity of Semaphorin 5A (protein) on immune cells in in vitro assays, it has been found that the addition of Semaphorin 5A to the cultivation medium increases the cytotoxic activity of NK cells, the cytotoxic activity of CD8⁺ T-cells, and the proliferation of CD8⁺ T-cells, also in the absence of target cells, e.g. of allogeneic cells. Accordingly, in the second embodiment, the invention provides Semaphorin 5A in a pharmaceutical composition or as an ingredient in a medicament for use in the activation of the immune system, especially for activating the cytotoxic activity of NK cells, for activating the cytotoxic activity of CD8⁺ T-cells, and for the proliferation of CD8⁺ T-cells. Preferably, the use of Semaphorin 5A is in the treatment of an insufficient immune system. The use of Semaphorin 5A as a medicament for the treatment of a low activity of the immune system, and Semaphorin 5A for use in the preparation of a pharmaceutical composition for use in the treatment of a low activity of the immune system, e.g. for increasing the activity of the immune system, preferably in patients suffering from congenital or acquired immune deficiencies, infectious diseases or cancer.

More preferably, the Semaphorin 5A for use as a medicament is a combination of Semaphorin 5A with IL-2 and/or with IL-15 for use as a medicament, especially for activating the immune system.

### Example 1

### Semaphorin 5A is Specifically Increased in GvHD Patients and HvGD Patients

After written consent, as applicable, peripheral blood mononuclear cell samples were isolated from 60 patients (30-60 years old) who underwent allogeneic hematopoietic stem cell therapy. Prior to this therapy, similar conditioning regimens were applied. From these patients, 5 were suspected of GvH disease, 6 patients presented clinical signs of GvH disease at the time the analysis was made, or had been diagnosed with GvH disease up to two months before, and a further 6 patients had been diagnosed with clinical GvH disease at 6-2 months before the analysis was made. 43 of the patients never developed GvH disease or were free of GvH disease for more then 6 months. As a control, PBMCs from 10 healthy individuals was used.

Total RNA was isolated from PBMC samples, and reverse transcription was performed on RNA to produce complementary DNA (cDNA) using a high capacity cDNA reverse transcription kit (Applied Biosystems, Foster City, USA). For each reaction, 50 ng total RNA was used. Semaphorin 5A and, in parallel reactions, Semaphorin 3A and β-actin mRNA were determined using a two-step real time PCR (TaqMan Gene Expression Master Mix, Applied Biosystems, Foster City, USA) using amplification primers and FAM-labelled minor groove binding (MGB) Taqman probes.

For Semaphorin 5A, the primers corresponding to SEQ ID NO 1 (5′-CAACGGCGGCCACTATG-3′) and to SEQ ID NO 2 (5′-CCAGGCGGGCTTTGC-3′), using MGB-probe of SEQ ID NO 3 (5′FAM-CAACGATTCCGATACACA 3′) were used, for Semaphorin 3A primers having SEQ ID NO 4 (5′-TTCAGCAATGGAGCTTTCCA-3′) and SEQ ID NO 5 (5′-CAACCCCAGCCGTTGAAC-3′) with the MGB-probe according to SEQ ID NO 6 (5′-FAM-AAGCAGCAACAACTATATA-3′) were used, and for β-actin primers according to SEQ ID NO 7 (5′-ATG ATG ATA TCG CCG CGC-3′) and SEQ ID NO 8 (5′-GCC TTG CAC ATG CCG G-3′), and MGB-probe having SEQ ID NO 9 (5′-FAM-CGT CGT CGA CAA CGG-3′) were used. For standardizing levels of Semaphorin 5A and of Semaphorin 3A mRNAs, respectively, the specific amount of β-actin-mRNA of parallel aliquots was used. Thermal cycling in a Step One plus real-time PCR system (Applied Biosystems) was 95° C. for 10 min, followed by 40 cycles at 95° C. for 15 s and 60° C. for 1 min. For analysis, the Step One software version 2.0 (Applied BioSystems) and a quantification-comparative method were used. Generally, patient data for Semaphorin 5A were normalized to the Semaphorin 5A signals of the healthy control group after normalizing each signal of Semaphorin 5A to that of β-actin.

The mRNA for human Semaphorin 5A is available under accession number NM_{—}003966.2 from GenBank (SEQ ID NO 10). Preferably, the nucleotide sequence of Semaphorin 5A corresponds to SEQ ID NO 10, and the specific identification of Semaphorin 5A uses a nucleotide sequence according to SEQ ID NO 10, e.g. contained in a sample and used in the above described RT-PCR. Further, Semaphorin 5A protein according to this invention is preferably encoded by nucleotide sequence according SEQ ID NO 10, more preferably from nt 779 to nt 3934, and most preferably, Semaphorin 5A protein according to this invention has an amino acid sequence according to SEQ ID NO 11.

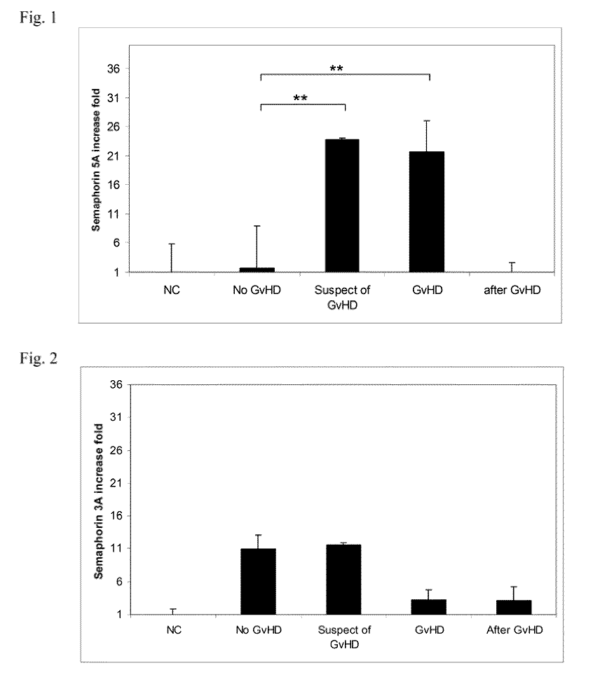

It was found that in comparison to the group without GvH disease (relative quantification (RQ):1.7±7.2), the patients suspected of GvH disease (RQ: 23.7±0.33; p<0.001) and patients with clinical diagnosis of GvH disease (RQ: 21.7±5.3; p<0.001) showed a significant increase of mRNA levels specific for Semaphorin 5A. After hematopoietic stem cell transplantation, no significant difference in the levels of Semaphorin 5A mRNA was observed in the group of patients after GvH disease (RQ: 1±1.7), when compared to the group without GvH disease. Results are shown in FIG. 1, wherein mRNA levels of Semaphorin 5A are normalized to β-actin mRNA levels and the levels of Semaphorin 5A mRNA are normalized to the values of the healthy control group. The data show that the level of Semaphorin 5A in PBMCs is indicative of the increased activity of the immune system occurring in GvH disease. In detail, the group of patients which are suspected of GvH disease (Suspect of GvH) and the group of patients being diagnosed with GvH disease (GvHD) have an increase in the normalized mRNA levels of Semaphorin 5A by a factor of about 20 in comparison to the control group (NC), and in relation to the patients without GvH disease (No GvHD). After treatment of the patients suffering from GvH disease (after GvHD), i.e. by standard immunosuppressant therapy, levels of Semaphorin 5A were in the same range as those of healthy control and no GvHD-patients, demonstrating the diagnostic value of Semaphorin 5A levels for GvH disease.

The analysis of Semaphorin 3A in the same patient samples showed that Semaphorin 5A was upregulated in GvH disease-cases, whereas Semaphorin 3A was not. In detail, for Semaphorin 3A, no difference was measured between patients without GvH disease (No GvHD, RQ: 10.9±2.25) and patients suspected of GvH disease (Suspect of GvHD, RQ: 11.5±0.4), and no differences were observed in patients diagnosed with GvH disease (GvHD, RQ: 3.2±1.59) and patients after GvH disease (After GvHD, RQ: 3.1±2.1), which results are shown in greater detail in FIG. 2.

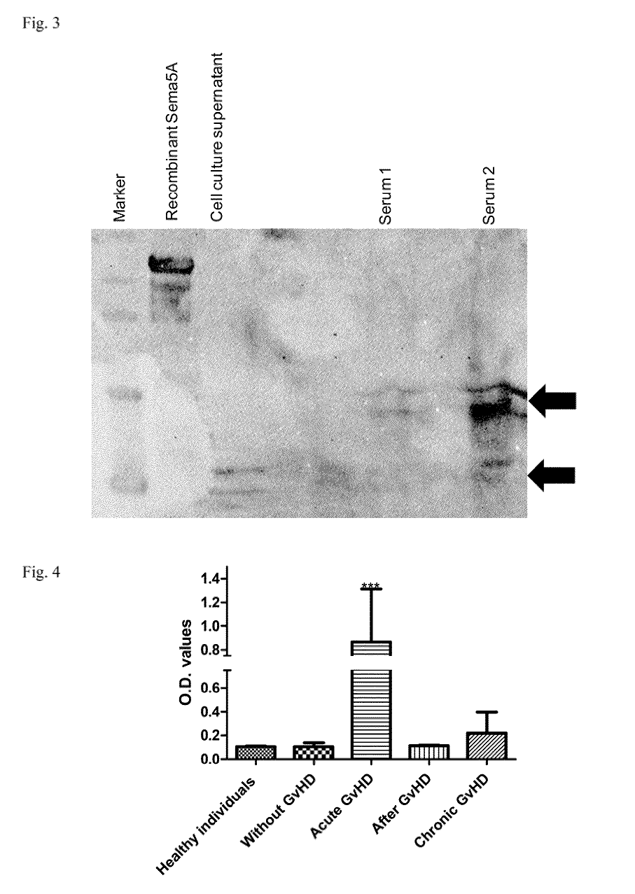

SEMA5A, i.e. Semaphorin 5A protein, was determined in an ELISA using a rabbit antibody that is specific for the N-terminal region of SEMA5A (available from Abcam), and an anti-rabbit antibody labelled by coupling to horse-radish peroxidase (HRP) as a secondary antibody. A Western blot analysis using the antibody (Abeam) specific for SEMA5A revealed that a soluble form of SEMA5A is present in sera of patients having GvHD and rheumatoid arthritis. The Western blot, done by SDS-PAGE and electrotransfer onto a nitrocellulose membrane according to standard procedures is shown in FIG. 3. For control, recombinant Semaphorin 5A protein, cell culture supernatant from cultivated HEK293 cells overexpressing human SEMA5A were used (3 lanes from the left, one empty lane), a serum from a rheumatoid arthritis patient (Serum 1), and a serum from a GvHD patient (Serum 2). As both patients were free from cancer, this analysis shows that the soluble form of SEMA5A can be detected in sera of patients having an undesired immune response, which is exemplified by GvHD and the autoimmune disease.

Sera were collected from a group of patients (n=27) prior to transplantation (HSCT), without GvHD after transplantation (n=24), for suspect or confirmed GvHD (n=3) after GvHD episodes (n=3), or from patients transplanted for more than 3 years with chronic GvHD (n=10). Sera from 10 healthy individuals served as a control. Results are shown as OD of the ELISA in FIG. 4, indicating a significant increase of SEMA5A scrum concentration in patients with suspected or diagnosed acute GvHD (0.867±0.446; p<0.001) in comparison to the concentrations determined in the sera of patients after HSCT who were free of GvHD (0.106±0.003), or in sera from those HSCT patients after being treated for GvHD (0.114±0.005). These results clearly indicate that SEMA5A concentrations are upregulated specifically in case of acute GvHD. A separate group of patients suffering of chronic GvHD was analysed. A non-significant increase of SEMA5A concentrations was observed (0.219±0.178). However, the chronic patients presented other severe malignancies such as lung or thyroid cancer and were under therapy at the time the sample was taken. A correlation of the SEMA5A concentrations from these patients with GvHD would therefore be hindered by the impact of anti-cancer therapy.

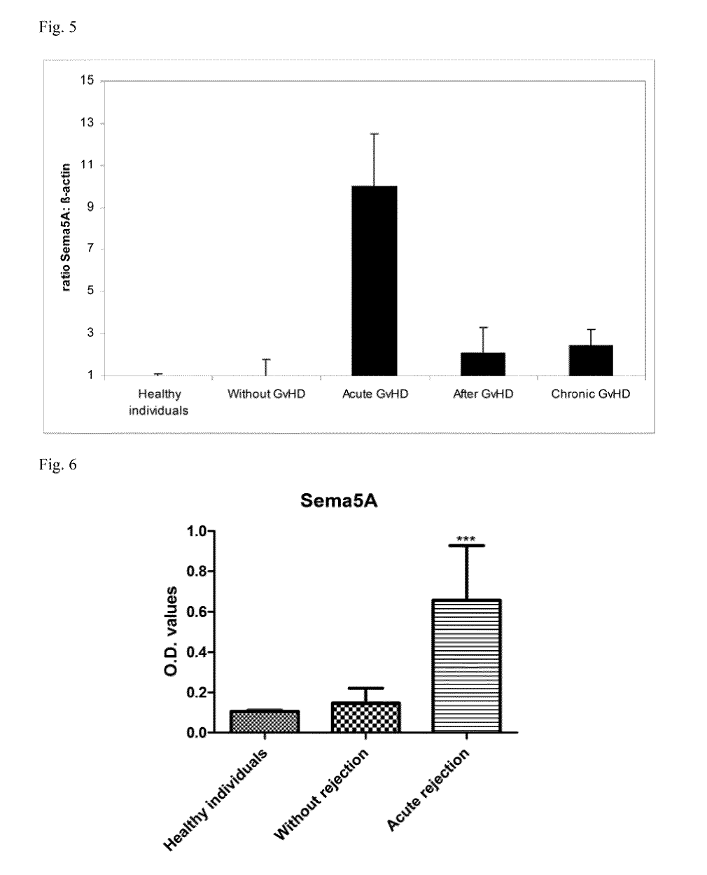

In addition, concentrations of mRNA encoding Semaphorin 5A (Sema5A mRNA) were detected by real-time PCR as described above. As shown for SEMA5A, elevated concentrations in the Sema5A mRNA in patients correlate with suspect or diagnosed GvHD (RQ 10±2.5, p<0.05) in comparison to Sema5A mRNA concentrations in patients after HSCT without GvHD (RQ 1±0.8) or after GvHD treatment (RQ 2.1±1.2). The group of patients showing chronic GvHD showed a RQ mean of 2.4±0.8. The results are shown in FIG. 5.

As a further example of transplant recipients, in which elevated levels of Sema5A mRNA or SEMA5A indicate an undesired immune response, e.g. the host versus graft disease (HvGD), samples from patients having received an allogeneic kidney transplant were analysed. Results are shown in FIG. 6 as OD obtained in the ELISA specific for SEMA5A, wherein *** indicates a significance p<0.001 in the one-way ANOVA statistical test followed by the post-hoc Bonferroni test. Sera from healthy individuals were used as control. This analysis makes it clear that also in the situation of solid organ transplantations, elevated concentrations of Semaphorin 5A indicate an increased activation of the immune system of the transplant recipient, i.e. HvGD, whereas controls and sera from transplant recipients who were free from HvGD, i.e. acute transplant rejection (Acute rejection) were analysed to have significantly lower serum concentrations of SEMA5A.

### Example 2

### Semaphorin 5A is Specifically Increased in Patients Having an Autoimmune Disease

As an example for autoimmune diseases, patients who were diagnosed with rheumatoid arthritis were used. In detail, SEMA5A concentrations were assessed by ELISA in sera from 15 rheumatoid arthritis (RA) patients prior any kind of therapy, and in 10 healthy individuals. Significantly increased SEMA5A concentrations were detected in RA patients (OD 0.380±0.182, p<0.001) compared to the levels observed in healthy individuals (OD 0.117±0.009). The results are shown in FIG. 7, wherein *** indicates a significance p<0.001 in the one-way ANOVA statistical test followed by the post-hoc Bonferroni test.

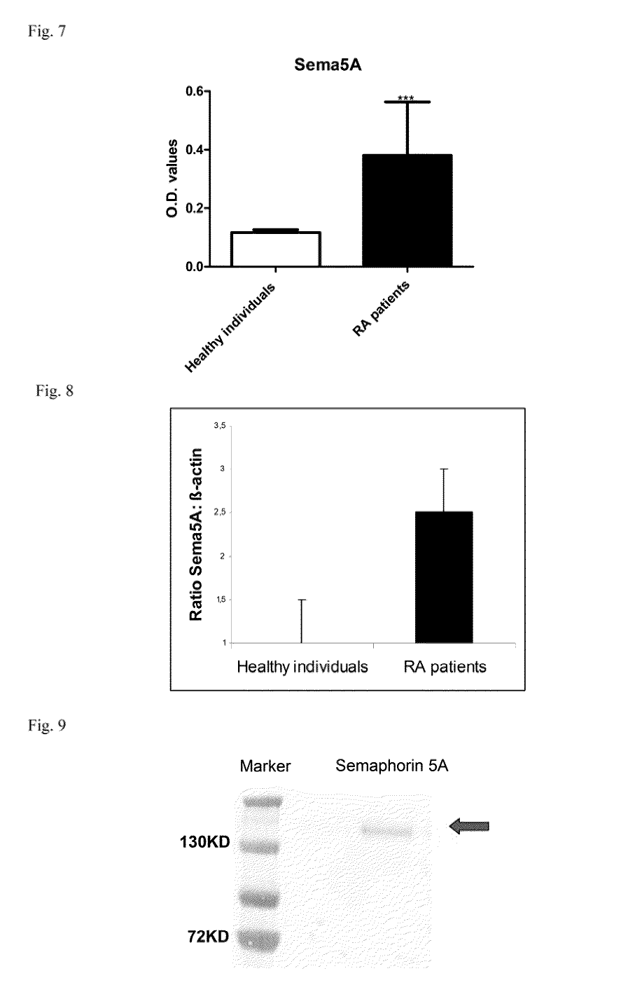

Determination of Sema5A mRNA concentrations in PBMCs enriched from whole blood of patients suffering of rheumatoid arthritis showed a significantly higher Sema5A mRNA concentration (RQ 2.511.2) in comparison to the concentrations measured in healthy individuals. Results are shown in FIG. 8, wherein Sema5A mRNA concentrations are normalized to the concentration of mRNA of the housekeeping gene β-actin, which was detected in parallel by quantitative RT-PCR.

### Example 3

### Production of Recombinant Semaphorin 5A

As an example for the manufacture of Semaphorin 5A, e.g. for use in the production of a pharmaceutical composition containing Semaphorin 5A for use in the treatment of a low activity of the immune system, the extracellular domain of human Semaphorin 5A was expressed in HEK cells. For ease of purification, the expression construct included a His-tag.

Briefly, the plasmid pcDNA3.1 was provided with the coding sequence for the extracellular domain of human Semaphorin 5A with an added soluble His-tag, and transfected using lipofection (Lipofectamine 2000, Invitrogen) into the human embryonic kidney 293 (HEK293) cell line. The recombinant Semaphorin 5A protein with its added His-tag was isolated from cell culture supernatant by immobilized-metal affinity chromatography on HisTrapFF columns (GE Healthcare, Little Chalfont, Great Britain). Cell culture supernatants were pooled, adjusted to pH 8.0, and loaded onto the columns using a BioLogic DuoFlow System, Bio-Rad, Hercules, USA). Elution of Semaphorin 5A was by 20 mM sodium phosphate, 0.5 M sodium chlorate, 0.5 M imidazole, pH 7.4. Recombinant protein was quality-controlled by Western blotting and an ELISA using detection antibodies against the V5 epitope and the His-tag (available from Invitrogen). The result is shown in FIG. 9, with the arrow indicating Semaphorin 5A.

### Example 4

### Production of an Antibody Having Specificity Against Semaphorin 5A

Polyclonal and monoclonal antibody specific for Semaphorin 5A was produced according to standard practice in experimental animals. In short, experimental mice were vaccinated with Semaphorin 5A as produced according to example 2, using at least 2 booster vaccinations. For polyclonal antibody, serum was collected and purified as generally known. The specificity could be determined against immobilized Semaphorin 5A by detecting bound antibody using a secondary antibody.

For monoclonal antibody, spleen cells of a mouse were used for the hybridoma technique, and from hybridoma cultures, a producer was screened according to the affinity of the supernatant to immobilized Semaphorin 5A.

### Example 5

### Activation of NK Cells and of the Cytotoxic Activity of NK Cells by Semaphorin 5A Protein

Natural killer cells (NK cells) were isolated from peripheral blood of 8 healthy donors using magnetic assisted cell separation technology. Cells were cultured in the presence of IL-2 and IL-15, in the presence of Semaphorin 5A alone, or in a combination of Semaphorin 5A with IL-2 and IL-15. As a control, heat-denatured Semaphorin 5A was used. Cultivation was generally in standard cell culture medium under standard cultivation conditions.

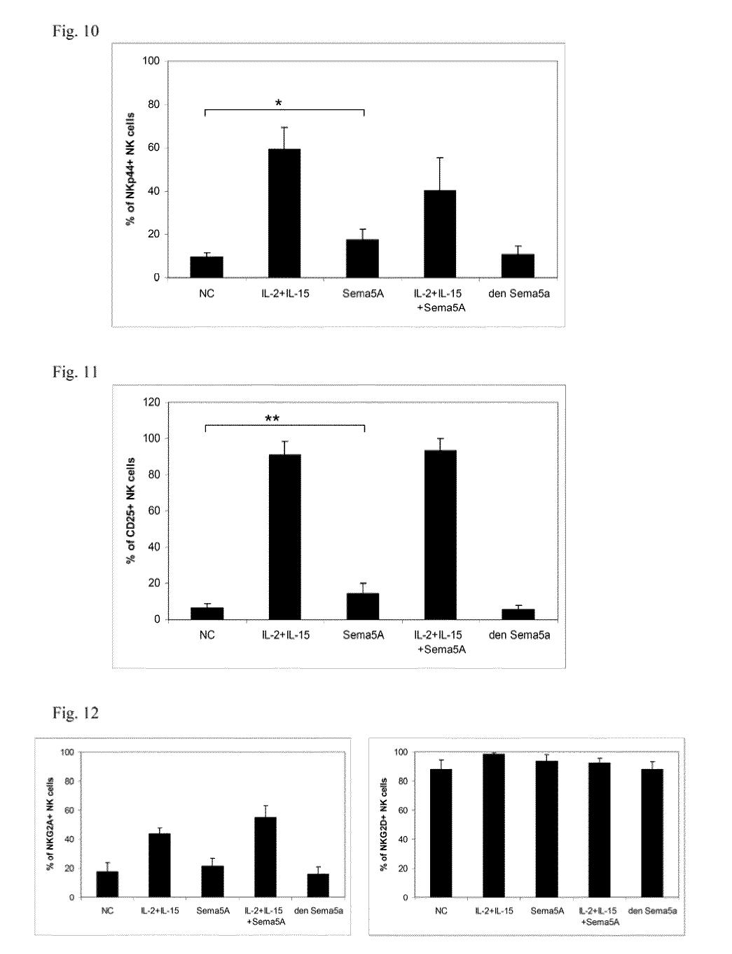

After 48 hours of cultivation, the following cell surface receptors were determined by flow cytometry specific for cell surface expression of KIR2DL1, KIR2DL2, NKp30, NKp44, NKp46, NKG2A, NKG2D, and CD25. Results are shown in FIG. 10 and demonstrate, that the exposure of the immune cells to Semaphorin 5A protein (Sema5A) leads to an increase of the natural cytotoxic receptor NKp44, i.e. NKp44+NK cells by approximately 12%. As expression of NKp44 is restricted to activated NK cells, this result demonstrates that Semaphorin 5A protein activates NK cells. This activation of NK cells is even more pronounced for the combination of Semaphorin 5A protein with IL-2 and IL-15 (IL-2+IL-15+Sema5A), whereas control cells (NC) without Semaphorin 5A and cells exposed to denatured Semaphorin 5A protein (den Sema5a) show lower activation.

In addition, exposure of the cultivated immune cells to Semaphorin 5A stimulated an increase of the frequency of CD25⁺ NK cells by up to 20%, as shown in FIG. 11. The stimulation is even more pronounced when Semaphorin 5A is used in combination with IL-2 and IL-15.

As shown in FIG. 12, the NKG2A+(21.2%±5.7%) and NKG2D+(93.8%±4%) NK cells increased significantly in the presence of Semaphorin 5A, when compared to NK cells that had been cultivated in the absence of Semaphorin 5A (NC), or NK cells that were incubated in the presence of Semaphorin 5A alone. No significant changes in the expression were observed for expression of NKp30, NKp46, KIR2DL1 or KIR2DL2 (data not shown).

This data show that Semaphorin 5A, e.g. in the form of recombinant Semaphorin 5A, and preferably in the presence of additional IL-2 and/or IL-15, is an activator of NK cell function.

As shown in FIG. 13, Semaphorin 5A stimulates NK cells to have increased levels of Granzyme B (1.72±0.31) in comparison to NK cells cultivated without Semaphorin 5A (1+0.05). mRNA levels of Granzyme B were evaluated by real-time PCR for determining its upregulation. The data shown are normalized to β-actin levels determined in aliquots.

An increased, e.g. synergistic effect for the upregulation of Granzyme B transcripts was observed for Semaphorin 5A in combination with both IL-2 and IL-15 (11.57±4.11), when compared to NK cells cultivated in IL-2 and IL-15 but without Semaphorin 5A (6.7±2.5). This clearly shows that Semaphorin 5A enhances the cytotoxic potential of NK cells, even in the absence of target cells. As a control, non-simulated NK cells were used.

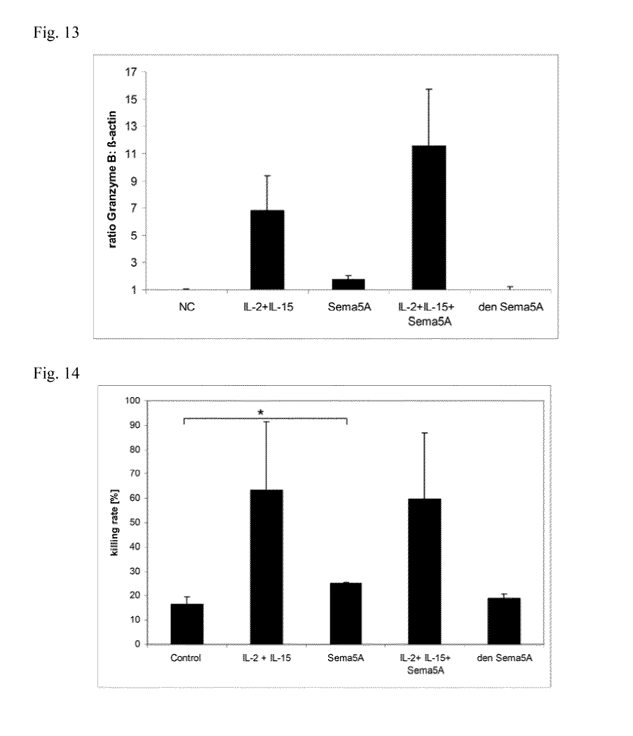

In an additional experiment, the NK cells previously incubated in the presence or absence of Semaphorin 5A, as described above, were used in cytotoxicity assays with K562 cells as targets. NK cells previously cultivated in the presence of Semaphorin 5A showed an increased lysing capacity of the target cells (25%±0.3%) in comparison to NK cells cultivated in the absence of Semaphorin 5A (16.5%±3%), as is also shown in FIG. 14.

### Example 5

### Stimulation of Proliferation of CD8⁺ T-Cells, and Increase of Cytotoxicity of CD8⁺ T-Cells by SEMAPHORIN 5A

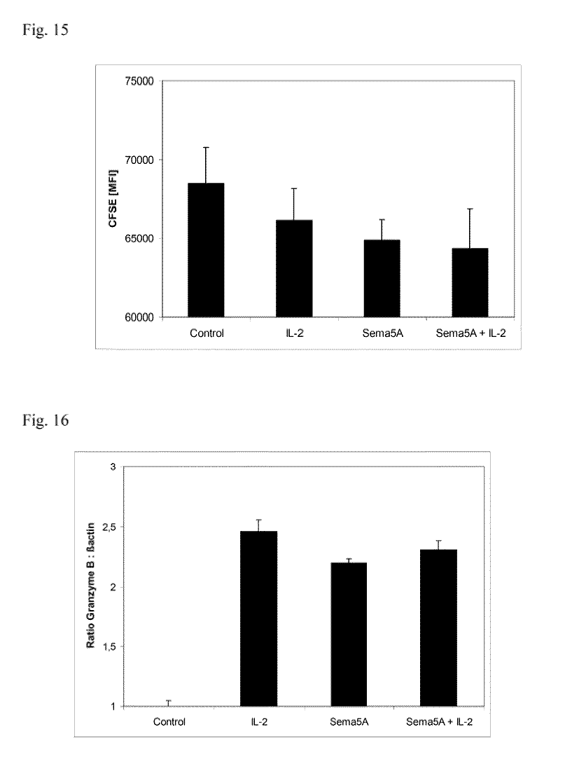

CD8⁺ T-cells were isolated from the peripheral blood of 4 healthy donors using magnetic assisted cell separation. Prior to the proliferation assays, CD8⁺ T-cells were labelled with 5,6-carboxy-succinimidyl-fluorescein-ester (CFSE), and cultured in the presence of IL-2 (50 U), Semaphorin 5A (10 μg) alone, or in a combination of Semaphorin 5A (10 μg) and IL-2 (50 U) for 5 days under standard cell cultivation conditions. As negative control in this experiment, non-stimulated cells (non-stimulated) were used, as NK cells are unable to endocytose protein and to produce cytokines in response thereto. In comparison to CD8⁺ T-cells cultivated without added stimulant (Control), cultures supplemented with IL-2 showed a 4% increase in the proliferation rate for CDC T-cells. A strong increase of the proliferation rate by about 10% was observed in the culture of CD8⁺ T-cells in the presence of Semaphorin 5A. Essentially no synergistic effect onto the proliferation rate was detected for the combination of IL-2 and Semaphorin 5A in this culture of CD8⁺ T-cells. Data are shown in FIG. 15.

For an evaluation of the cytotoxic potential of CD8⁺ T-cells, total RNA was isolated from the individual cultures, and the level of Granzyme B transcripts were measured by real-time PCR. Data are shown in FIG. 16. Presence of IL-2 in the cultivation of CD8⁺ T-cells caused a 2.4±0.1-fold increase in levels of Granzyme B. Presence of Semaphorin 5A alone, i.e. without additional IL-2, also induces an upregulation of Granzyme B-transcripts by a factor of 2.2±0.1. The combination added IL-2 and Semaphorin 5A lead to an increase of levels of Granzyme B by a factor of 2.3±0.07. These data demonstrate that it is Semaphorin 5A alone that is capable of inducing cell proliferation and an increased cytotoxicity of CD8⁺ T-cells.

Cytokines, which are known to play a crucial role in inflammatory diseases, graft rejection and GvHD were analysed by determination of the cytokine secretion profile of NK cells and monocytes obtained from healthy individuals upon addition of SEMA5A. Freshly isolated NK cells from five healthy donors were stimulated with 10 μg/ml of recombinant SEMA5A in presence or absence of a combination of IL-2 (100 U/mL) plus IL-15 (50 ng/mL). After 2 days of culture under standard conditions, the NK cell culture supernatant was harvested and analysed for the presence of cytokines using the Luminex technology (available from Invitrogen). A significant increase in the secretion of IL-1β, IL-6 and IL-8 was detected upon stimulation of NK cells with SEMA5A in comparison to the cytokine secretion profile of non-stimulated NK cells. Furthermore, a significantly increase in the secretion of IFN-γ, IL-1β; IL-6, IL-8, TNF-α, GM-CSF was measured when NK cells were incubated in the presence of SEMA5A in combination with IL-2 and IL-15 in comparison to the cytokine levels detected in the presence of this combination of cytokines alone. Results are summarized in Table 1. No differences were observed in the secretion levels of IL-4, IL-5, IL-7, IL-10, IL-12(p70), IL-13 and MCP-1.

TABLE 1
Cytokine secretion profile of NK cells incubated in the presence or absence of Sema5A
Added stimulant

| Analytes | Non- | | | |
|---|---|---|---|---|
| (pg/ml) | stimulated | IL-2 + IL-15 | Sema5A | Sema5A + IL-2 + IL-15 |
| IFN-γ | 0 | 209.07 ± 141.70 | 43.56 ± 52.09 | 15471 ± 4013.35 |
| | | | | (p < 0.001) |
| IL-1β | 0 | 0 | 50.90 ± 2.01 | 70.10 ± 8.45 |
| | | | (p < 0.01) | (p < 0.01) |
| IL-6 | 6.66 ± o.94 | 10.66 ± 3.12 | 303.39 ± 54.13 | 437.51 ± 0.76 |
| | | | (p < 0.01) | (p < 0.001) |
| IL-8 | 52.12 ± 44.32 | 1241.24 ± 486.12 | 4196.90 ± 1258.05 | 6666.29 ± 3932 ± 93 |
| | | | (p < 0.001) | (p < 0.001) |
| TNF-α | 3.64 ± 0.1 | 6.89 ± 1.32 | 6.66 ± 1.65 | 18.83 ± 8.96 |
| | | | | (p < 0.01) |
| GM-CSF | 0 | 179.46 ± 111.36 | 33.36 ± 6.94 | 523.65 ± 112.9 |
| | | | | (p < 0.01) |

Further, the cytokine secretion profile of monocytes in response to SEMA5A was analysed. CD 14+ cells were isolated from PBMCs of 6 healthy individuals and stimulated in presence of 10 μg/mL of recombinant SEMA5A protein alone or in presence of 10 μg/ml of recombinant SEMA5A in combination with 50 ng/ml IFN-γ. A soluble form of a killer immunoglobulin-like receptor (KIR) protein was used as negative control. After 2 days cultivation under standard conditions, monocyte cell culture supernatants were harvested and analysed for the presence of cytokines using Luminex technology (available from Invitrogen). A significant increase in the secretion of IL-1β was observed when monocytes were stimulated in the presence of SEMA5A protein alone (3149.095±2366.675 pg/mL; p<0.05) or in combination with IFN-γ (6083.278±2364.507 pg/mL; p<0.05) in comparison to the levels detected in non-stimulated monocyte cultures (1227.193±660.273 pg/mL) or in presence of IFN-γ (4474.031±3429.2 pg/mL), respectively. Also, exposure of monocytes to a combination of SEMA5A and IFN-γ caused an augmentation in the secretion of IL-12(p70) (392.41±200.153 pg/mL; p<0.05). In addition, stimulation of monocytes with SEMA5A protein alone (3028.207±2321.318 pg/mL; p<0.01) or in combination with IFN-γ

TABLE 2
Cytokine secretion profile of monocytes in presence or absence of SEMA5A
added stimulant

| Analytes | | | | | | NC + |
|---|---|---|---|---|---|---|
| (pg/mL) | Non-stimulated | IFN-γ | SEMA5A | SEMA5A + IFN-γ | NC | IFN-γ |
| IL-1β | 1227.193 ± 660.273 | 4474.031 ± 3429.2 | 3149.095 ± 2366.675 | 6083.278 ± 2364.507 | 343.2 ± 230.1 | 3900.2 ± 859.1 |
| | | | (p < 0.05) | (p < 0.05) | | |
| IL-12(p70) | 9.467 ± 3.225 | 173.029 ± 174.819 | 11.714 ± 5.386 | 392.41 ± 200.153 | 3.2 ± 0.1 | 113.2 ± 20.1 |
| | | | | (p < 0.05) | | |
| TNF-α | 1547.655 ± 743.138 | 9036.502 ± 2321.318 | 3028.207 ± 2321.318 | 11075.343 ± 806.811 | 8.823 ± 3.596 | 7930.918 ± 2645.176 |
| | | | (p < 0.01) | (p < 0.05) | | |

(11075.343 ± 806.811 pg/mL; p < 0.05) resulted in a significant increase of TNF-α secretion levels in comparison to non-stimulated monocytes (1547.655 ± 743.138 pg/mL) or in presence of IFN-γ (9036.502 ± 2321.318 pg/mL). Results are summarized in Table 2, wherein NC indicates absence of SEMA5A. As a negative control (NC) in this experiment, monocytes were stimulated with another protein than SEMA5A in the presence or absence of IFN-γ in addition tonon-stimulated cells (non-stimulated).

The comparison of the NC to the specific effect of SEMA5A shows that the observed effect was caused by SEMA5A, excluding the possibility that variations in the monocyte secretion profile were a result of endocytosis of protein.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A method for analysis of the state of activation of the immune system of a patient comprising determination of the concentration of Semaphorin 5A in a sample of the patient.

2. The method according to claim 1, wherein the concentration of Semaphorin 5A is determined by detecting the mRNA encoding Semaphorin 5A, or by detecting the reaction of an antibody that is specific for Semaphorin 5A protein.

3. The method according to claim 2, wherein the concentration of Semaphorin 5A is normalized to a signal determined for a constitutively expressed constituent of the sample.

4. The method according to claim 1, wherein the determined concentration of Semaphorin 5A is compared to the concentration of Semaphorin 5A determined in a healthy human, wherein an elevated concentration of Semaphorin 5A in the sample of the patient is indicative of an activated state of the patient's immune system.

5. The method according to claim 1, wherein the patient is a recipient of an allogeneic transplant or is diagnosed as having or is suspected of having an autoimmune disease, excluding cancer patients.

6. The method according to claim 4, wherein the state of activation of the immune system is used to indicate the presence of GvHD or HvGD.

7. A Kit of components for use in a method according to claim 1, comprising a pair of oligonucleotides specifically hybridising to a nucleotide sequence containing at least a fraction of the nucleotide sequence encoding Semaphorin 5A.

8. A kit according to claim 7, further comprising a pair of oligonucleotides specifically hybridising to a nucleotide sequence encoding at least a fraction of a constitutively expressed mammalian protein.

9. A kit of components for use in a method according to one of claim 1, comprising an antibody which is specific for Semaphorin 5A protein.

10. A kit according to claim 9, further comprising an antibody which is specific for a constitutively expressed mammalian protein.

11. A medicament for activating the immune system in a patient comprising Semaphorin 5A protein in a pharmaceutically acceptable formulation.

12. The medicament according to claim 11, wherein the medicament is provided for a patient who is free from cancer.

13. The medicament according to claim 11, wherein the Semaphorin 5A protein has an amino acid sequence according to SEQ ID NO 11.

14. The medicament according to claim 11, wherein the Semaphorin 5A protein is in combination with IL-2 and/or IL-15.

15. A process for the production of a pharmaceutical composition which is for use in the activation of the immune system of a patient, excluding cancer patients, by providing Semaphorin 5A protein in a pharmaceutically acceptable formulation.

16. The process according to claim 15, wherein the pharmaceutically acceptable formulation is suitable for i.v. injection purposes.
